SEQUENCE LISTING (4 sequences)

SEQ ID NO: 1 - DNA, 21 bp, synthetic construct
  source 1..21: mol_type = other DNA
  modified_base 5: mod_base = OTHER; note = uracil
  modified_base 7: mod_base = OTHER; note = uracil
  modified_base 10: mod_base = OTHER; note = uracil
  modified_base 16: mod_base = OTHER; note = uracil
cgggtatggtggagctaaatt

SEQ ID NO: 2 - DNA, 21 bp, synthetic construct
  source 1..21: mol_type = other DNA
  modified_base 4: mod_base = OTHER; note = uracil
  modified_base 6: mod_base = OTHER; note = uracil
  modified_base 13: mod_base = OTHER; note = uracil
  modified_base 14: mod_base = OTHER; note = uracil
ggatataagcagttgaggatt

SEQ ID NO: 3 - DNA, 16 bp, synthetic construct
  source 1..16: mol_type = other DNA
gcgagtaagcagaaga

SEQ ID NO: 4 - DNA, 16 bp, synthetic construct
  source 1..16: mol_type = other DNA
gaaagggtgtcacagt

(19) United States
(12) Patent Application Publication
(10) Pub. No.: US 2026/0286359 A1
(43) Pub. Date: Sep. 24, 2026

(54) LONG NON-CODING RNA AND CDK4/6 INHIBITORS IN BREAST CANCER

(71) Applicant: Washington University, St. Louis, MO (US)

(72) Inventors: Jessica Silva-Fisher, St. Louis, MO (US); Richa Mishra, St. Louis, MO (US)

(21) Appl. No.: 19/575,036

(22) Filed: Mar. 23, 2026

Related U.S. Application Data
(60) Provisional application No. 63/776,704, filed on Mar. 24, 2025.

(51) Int. Cl. C12N 15/113; A61K 31/506; A61K 31/519

(52) U.S. Cl. CPC C12N 15/113; A61K 31/506; A61K 31/519; C12N 2310/11; C12N 2310/3231

(57) ABSTRACT

The present teachings include compositions comprising a locked nucleic acid-based antisense oligonucleotide (LNA ASO) targeting LINC00355 and methods of using these compositions for treating cancer.

### CROSS-REFERENCE TO RELATED APPLICATIONS

This application claims the benefit under 35 U.S.C. § 119 (e) of U.S. Provisional Patent Application No. 63/776,704, filed Mar. 24, 2025, the entire disclosure of which is incorporated herein by reference.

### REFERENCE TO SEQUENCE LISTING

This application includes a Sequence Listing filed electronically as an XML file named 150602137SEQ, created on May 5, 2026, with a size of 8,803 bytes. The Sequence Listing is incorporated herein by reference.

### FIELD OF THE INVENTION

The present disclosure generally relates to methods and compositions for treating cancer.

### BACKGROUND OF THE INVENTION

Breast cancer is the most common cancer diagnosed among women in the United States, with approximately 310,720 new cases estimated in 2024. Late relapse refers to disease recurrence after being disease free for at least five years following the initial curative treatment. Late relapse is particularly relevant to ER+/HER2-breast cancer as it accounts for over 50% of recurrences for patients with this subtype of breast cancer. Cyclin-dependent kinase 4 and 6 (CDK4/6) inhibitors in combination with endocrine therapy are standard first line therapy at metastatic relapse. While these therapies initially show promise, resistance to endocrine therapy and CDK4/6 inhibitors inevitably develops, presenting a major clinical challenge. Long non-coding RNAs (lncRNAs) are key regulators of cancer-related processes, including cell cycle progression. Previous studies have identified deregulated lncRNAs in metastatic late relapse breast cancer patient tumor samples, but their role in CDK4/6 inhibitor resistance remains unclear (Eteleeb et al., npj Breast Cancer, 2022, 8, 49). To investigate this, ER+ primary breast cancer cell line MCF7 (wild-type) and the long-term estrogen-deprived (LTED) cell line-mimicking late relapse-were treated with CDK4/6 inhibitors, including Abemaciclib, Palbociclib, and Ribociclib. Wild-type cells were more sensitive to these inhibitors than LTED cells. Further analysis identified LINC00355, the most upregulated lncRNA in late relapse breast cancer, as binding to the MENIN protein. This was demonstrated using RNA pulldown assays and multiplexed RNA in situ hybridization in LTED cells and patient samples. The LINC00355-MENIN interaction decreases CDKN1B expression, promoting cellular proliferation. Notably, MENIN has been shown to regulate the CDK4/6 pathway and inhibit cell proliferation. To explore therapeutic potential, LINC00355 expression was reduced using GapmeRs, locked nucleic acid-based antisense oligonucleotides (LNA ASOs), which significantly decreased cell proliferation. In contrast, treatment of LTED cells with Palbociclib or Ribociclib alone for 72 hours showed no significant changes in proliferation. However, combination treatment with LINC00355 LNA ASOs and Palbociclib or Ribociclib resulted in a notable reduction in cell proliferation. These findings highlight the critical role of lncRNA-protein interactions, such as LINC00355-MENIN, in promoting cellular proliferation and provide proof-of-concept for targeting lncRNAs that are resistant to CDK4/6 inhibitors for late relapse breast cancer.

### SUMMARY OF THE INVENTION

Among the various aspects of the present disclosure is the provision of methods and compositions for treating cancer.

Briefly, therefore, the present disclosure is directed to methods and compositions to specifically target LINC00355 in cancers such as late relapse breast cancer.

In one aspect, compositions for treating cancer comprise a locked nucleic acid-based antisense oligonucleotide (LNA ASO), wherein the locked nucleic acid-based antisense oligonucleotide targets LINC00355, are disclosed.

In various other aspects, the locked nucleic acid-based antisense oligonucleotide comprises SEQ ID NO:3 or SEQ ID NO:4.

In various other aspects, the composition further comprises a CDK4/6 inhibitor. In some aspects the CDK4/6 inhibitor is selected from the group consisting of Abemaciclib, Palbociclib, Ribociclib, or combinations thereof.

In various other aspects, a method of treating cancer in a subject is described that includes administering the locked nucleic acid-based antisense oligonucleotide (LNA ASO) composition described above.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 illustrates how LINC00355 binds to MENIN thereby regulating CDKN1B expression, increasing the S phase cell cycle, and increasing cellular proliferation and invasion.

FIG. 2A illustrates expression of Estrogen Receptor in primary and LTED cell lines.

FIG. 2B illustrates expression of LINC00355 in primary and LTED cell lines.

FIG. 3 illustrates LINC00355 expression in primary, early-stage relapse, and late-relapse PDX lines.

FIG. 4A illustrates RNA pull-down of LINC00355 with Menin in MCF7 LTED cells.

FIG. 4B illustrates LINC00355 RNA immunoprecipitation of MENIN compared to control gene and IgG.

FIG. 4C illustrates Wild-type (MCF7 WT) cells compared to MCF7 LTED cells cross-linking immunoprecipitation RT-qPCR with MENIN.

FIG. 4D illustrates co-detection of LINC00355 and MENIN in late-relapse breast tumor.

FIG. 5A illustrates reducing MENIN expression decreases cell proliferation. **p value <0.005.

FIG. 5B illustrates reducing MENIN expression decreases cell proliferation as measured by EdU incorporation. **p value <0.005.

FIG. 5C illustrates reducing MENIN expression decreases cell proliferation as measured by decreased S phase. **p value <0.005.

FIG. 6A illustrates LINC00355 expression in ASO treated cells.

FIG. 6B illustrates normalized cell viability six days post treatment. p value <0.0005.

FIG. 7A illustrates co-treatment of MCF7 LTED cells with CDK4/6 inhibitors and LINC00355-targeting LNA ASOs results in decreased proliferation.

FIG. 7B illustrates LINC00355 overexpression increases proliferation under CDK4/6 inhibitor treatment.

FIG. 8A illustrates expression of LINC00355 and CDK4/6 pathway genes when treating MCF7 LTED cells with palbociclib or ribociclib alone.

FIG. 8B illustrates expression of LINC00355 and CDK4/6 pathway genes when treating MCF7 LTED cells with palbociclib or ribociclib in combination with LINC00355 ASO.

FIG. 8C illustrates Western blot analysis showing reduced phosphorylation of retinoblastoma (RB) protein following combined CDK4/6 inhibition and LINC00355 knockdown. * p<0.05 or ≥2-fold change; * p<0.005 or ≥5-fold change; * p<0.0005.

FIG. 9 illustrates a model of LINC00355-MENIN regulation of CDK4/6 to increase cell proliferation.

### DETAILED DESCRIPTION OF THE INVENTION

Among the various aspects of the present disclosure are the provision of locked nucleic acid-based antisense oligonucleotide compositions targeting LINC00355 and methods of treating cancer using the disclosed compositions.

In some aspects, the locked nucleic acid-based antisense oligonucleotide compositions comprise a 5 to 25 nucleotide long oligonucleotide. In some aspects, the locked nucleic acid-based antisense oligonucleotide targeting LINC00355 comprises the nucleotide GCGAGTAAGCAGAAGA (SEQ ID NO:3), GAAAGGGTGTCACAGT (SEQ ID NO:4), or modifications thereof.

In some aspects, the locked nucleic acid-based antisense oligonucleotide composition further comprises a CDK4/6 inhibitor. In some aspects the CDK4/6 inhibitor is selected from the group consisting of Abemaciclib, Palbociclib, Ribociclib, or combinations thereof.

In another aspect, the present disclosure relates to a pharmaceutical composition comprising a locked nucleic acid-based antisense oligonucleotide composition that targets LINC00355, or a pharmaceutically acceptable salt thereof, and a pharmaceutically acceptable carrier.

Among other aspects of the present disclosure are the provision of silencer RNA compositions targeting LINC0035 and methods of treating cancer using the disclosed compositions.

In some aspects, the silencer RNA compositions comprise a 5 to 25 nucleotide long oligonucleotide. In some aspects, the silencer RNA targeting LINC00355 comprises the nucleotide CGGGUAUGGUGGAGCUAAAtt (SEQ ID NO: 1), GGAUAUAAGCAGUUGAGGAtt (SEQ ID NO:2), or modifications thereof.

In some aspects, the silencer RNA composition further comprises a CDK4/6 inhibitor. In some aspects the CDK4/6 inhibitor is selected from the group consisting of Abemaciclib, Palbociclib, Ribociclib, or combinations thereof.

In another aspect, the present disclosure relates to a pharmaceutical composition comprising a silencer RNA composition that targets LINC00355, or a pharmaceutically acceptable salt thereof, and a pharmaceutically acceptable carrier.

In various aspects, a method of treating cancer in a subject is described that includes administering the locked nucleic acid-based antisense oligonucleotide (LNA ASO) composition described above. In some aspects, the method further comprises administering a CDK4/6 inhibitor. In some aspects, the CDK4/6 inhibitor is administered prior to administering the therapeutically effective amount of a composition comprising a locked nucleic acid-based antisense oligonucleotide (LNA ASO). In other aspects, the CDK4/6 inhibitor is administered after administering the therapeutically effective amount of a composition comprising a locked nucleic acid-based antisense oligonucleotide (LNA ASO). In another aspect the CDK4/6 inhibitor and the therapeutically effective amount of a composition comprising a locked nucleic acid-based antisense oligonucleotide (LNA ASO) are administered simultaneously.

In various aspects, a method of treating cancer in a subject is described that includes administering the silencer RNA composition described above. In some aspects, the method further comprises administering a CDK4/6 inhibitor. In some aspects, the CDK4/6 inhibitor is administered prior to administering the therapeutically effective amount of a composition comprising a silencer RNA. In other aspects, the CDK4/6 inhibitor is administered after administering the therapeutically effective amount of a composition comprising a silencer RNA. In another aspect the CDK4/6 inhibitor and the therapeutically effective amount of a composition comprising a silencer RNA are administered simultaneously.

In certain aspects, the present disclosure relate to methods for the treatment of cancer in a subject comprising administering to a subject having cancer an effective amount of a RNAi inhibitor of LINC00355 lncRNA function and/or its expression from the LINC00355 gene. In some embodiments, the presence of cancer in the subject is indicated by high levels of LINC00355 lncRNA expression, for example, as measured by the methods and systems as disclosed herein and known in the art. In some embodiments, high levels of LINC00355 lncRNA are at least about 125-fold increased as compared to a reference LINC00355 lncRNA level. In some embodiments, high levels of LINC00355 lncRNA are between about a 125-fold and 2000-fold increase, or a greater than 2000-fold increase as compared to a reference LINC00355 lncRNA level.

One aspect of the present disclosure relates to methods for decreasing LINC00355 lncRNA level in a cancer cell, comprising contacting the cancer cell with a RNAi inhibitor of LINC00355 lncRNA function and/or a RNAi inhibitor of LINC00355 lncRNA expression from the LINC00355 gene. In some embodiments, a RNAi inhibitor of LINC00355 lncRNA function or its expression from the LINC00355 gene can be selected from the group consisting of siRNA, miRNA, stRNA, snRNA, and antisense oligonucleotides (ASO), and in some embodiments, can be a locked nucleic acid-based antisense oligonucleotide (LNA ASO) which targets LINC00355 lncRNA and/or its expression from the LINC00355 gene.

Another aspect of the present disclosure relates to a method for treating a cancer in a subject, comprising; (a) contacting a biological sample from the subject with at least one nucleic acid binding probe to measure the level of LINC00355 lncRNA in the biological sample; (b) comparing the level of LINC00355 lncRNA in the biological sample to a reference level of LINC00355 lncRNA from a biological sample from a healthy population, wherein an increased level of LINC00355 lncRNA in the biological sample from the subject compared to the reference level of LINC00355 lncRNA indicates likelihood of the subject having cancer; and (c) administering a RNAi inhibitor of LINC00355 lncRNA and/or its expression from the LINC00355 gene in an effective amount to inhibit cancer in the subject.

Another aspect of the present disclosure relates to a method for inhibiting invasive growth of a cancer cell that misexpresses LINC00355 comprising contacting said cell with a RNAi inhibitor that decreases LINC00355 lncRNA in said cell.

In another aspect of the present disclosure relates to a method for detecting a cancer in a subject, comprising; (a) contacting a biological sample from the subject with at least one nucleic acid binding probe to measure the level of LINC00355 lncRNA in the biological sample; (b) comparing the level of LINC00355 lncRNA in the biological sample to a reference level of LINC00355 lncRNA from a biological sample from a healthy population, wherein an increased level of LINC00355 lncRNA in the biological sample from the subject compared to the reference level of LINC00355 lncRNA indicates likelihood of the subject having cancer.

LINC00355 biomarker polynucleotides (e.g., lncRNAs) can be detected, for example, by microarray analysis, polymerase chain reaction (PCR), reverse transcriptase polymerase chain reaction (RT-PCR), Northern blot, serial analysis of gene expression (SAGE), immunoassay, or mass spectrometry.

In all aspects, the cancer is late relapse breast cancer.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, a “subject” is any organism or animal to whom which treatment or prophylaxis treatment is desired. Such animals include mammals, preferably a human. The term “subject” also refers to any living organism from which a biological sample can be obtained. The term includes, but is not limited to, humans, non-human primates such as chimpanzees and other apes and monkey species; farm animals such as cattle, sheep, pigs, goats and horses, domestic subjects such as dogs and cats, laboratory animals including rodents such as mice, rats and guinea pigs, and the like. The term does not denote a particular age or sex. Thus, adult and newborn subjects, as well as fetuses, whether male or female, are intended to be covered. The term “subject” is also intended to include living organisms susceptible to conditions or diseases caused or contributed bacteria, pathogens, disease states or conditions as generally disclosed, but not limited to, throughout this specification. Examples of subjects include humans, dogs, cats, cows, goats, and mice. The term subject is further intended to include transgenic species. In another embodiment, the subject is an experimental animal or animal substitute as a disease model.

The term “mammal” or “mammalian” are used interchangeably herein, are intended to encompass their normal meaning. While the invention is most desirably intended for efficacy in humans, it may also be employed in domestic mammals such as canines, felines, and equines, as well as in mammals of particular interest, e.g., zoo animals, farmstock, transgenic animals, rodents and the like.

As used herein, “gene silencing” or “gene silenced” in reference to an activity of a RNAi molecule, for example a LNA ASO, siRNA, miRNA refers to a decrease in the mRNA level in a cell for a heterologous target gene by at least about 5%, about 10%, about 20%, about 30%, about 40%, about 50%, about 60%, about 70%, about 80%, about 90%, about 95%, about 99%, or about 100% of the mRNA level found in the cell without the presence of the miRNA or RNA interference molecule. In one preferred embodiment, the mRNA levels are decreased by at least about 70%, about 80%, about 90%, about 95%, about 99%, or about 100%. As used herein, the “reduced” or “gene silencing” refers to lower, preferably significantly lower, more preferably the expression of the nucleotide sequence is not detectable.

The term “double-stranded RNA” molecule, “RNAi molecule”, or “dsRNA” molecule as used herein refers to a sense RNA fragment of a nucleotide sequence and an antisense RNA fragment of the nucleotide sequence, which both comprise nucleotide sequences complementary to one another, thereby allowing the sense and antisense RNA fragments to pair and form a double-stranded RNA molecule. In some embodiments, the terms refer to a double-stranded RNA molecule capable, when expressed, of at least partially reducing the level of the mRNA of the heterologous target gene. In particular, the RNAi molecule is complementary to a synthetic RNAi target sequence located in a non-coding region of the heterologous target gene. As used herein, “RNA interference”, “RNAi”, and “dsRNAi” are used interchangeably herein to refer to nucleic acid molecules capable of gene silencing.

As used herein, the term “RNAi” refers to any type of interfering RNA, including, but not limited to, LNA ASO, siRNAi, shRNAi, stRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “siRNA” also refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full length target gene, or a subsequence thereof. Typically, the siRNA is at least about 10-50 nucleotides in length (e.g., each complementary sequence of the double stranded siRNA is about 10-22 nucleotides in length, and the double stranded siRNA is about 10-22 base pairs in length, preferably about 19-22 base nucleotides, preferably about 17-19 nucleotides in length, e.g., 10, 11, 12, 13, 14, 15, 16, 17, 18, 19, 20, 21, or 22 nucleotides in length).

As used herein, “a reduction” of the level of a gene, means a decrease in the level of a protein or mRNA in the cell or organism. As used herein, “at least a partial reduction” of the level of an agent (such as a RNA, mRNA, rRNA, tRNA expressed by the target gene and/or of the protein product encoded by it) means that the level is reduced at least 25%, preferably at least 50%, relative to a cell or organism lacking the RNAi agent as disclosed herein. As used herein, “a substantial reduction” of the level of an agent such as a protein or mRNA means that the level is reduced relative to a cell or organism lacking a chimeric RNA molecule of the invention capable of reducing the agent, where the reduction of the level of the agent is at least 75%, preferably at least 85%. The reduction can be determined by methods with which the skilled worker is familiar. Thus, the reduction of the transgene protein can be determined for example by an immunological detection of the protein. Moreover, biochemical techniques such as Northern hybridization, nuclease protection assay, reverse transcription (quantitative RT-PCR), ELISA (enzyme-linked immunosorbent assay), Western blotting, radioimmunoassay (RIA) or other immunoassays and fluorescence-activated cell analysis (FACS) can be used to detect transgene protein or mRNA. Depending on the type of the reduced transgene, its activity or the effect on the phenotype of the organism or the cell may also be determined. Methods for determining the protein quantity are known to the skilled worker. Examples, which may be mentioned, are: the micro-Biuret method (Goa J (1953) Scand J Clin Lab Invest 5:218-222), the Folin-Ciocalteau method (Lowry O H et al. (1951) J Biol Chem 193:265-275) or measuring the absorption of CBB G-250 (Bradford M M (1976) Analyt Biochem 72:248-254).

The terms “malignancy” or “cancer” are used interchangeably herein and refers to any disease of an organ or tissue in mammals characterized by poorly controlled or uncontrolled multiplication of normal or abnormal cells in that tissue and its effect on the body as a whole. Cancer diseases within the scope of the definition comprise benign neoplasms, dysplasias, hyperplasias as well as neoplasms showing metastatic growth or any other transformations like e.g. leukoplakias which often precede a breakout of cancer. The term “tumor” or “tumor cell” are used interchangeably herein, refers to the tissue mass or tissue type of cell that is undergoing abnormal proliferation.

The term “biological sample” as used herein refers to a cell or population of cells or a quantity of tissue or fluid from a subject. Most often, the sample has been removed from a subject, but the term “biological sample” can also refer to cells or tissue analyzed in vivo, i.e. without removal from the subject. Often, a “biological sample” will contain cells from the animal, but the term can also refer to non-cellular biological material, such as non-cellular fractions of blood, saliva, or urine, that can be used to measure gene expression levels. Biological samples include, but are not limited to, tissue biopsies, scrapes (e.g. buccal scrapes), whole blood, plasma, serum, urine, saliva, cell culture, or cerebrospinal fluid. Biological samples also include tissue biopsies, or cell culture. A biological sample or tissue sample can refer to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, the sample is from a resection, bronchoscopic biopsy, or core needle biopsy of a primary or metastatic tumor, or a cellblock from pleural fluid. In addition, fine needle aspirate samples are used. Samples may be either paraffin-embedded or frozen tissue. The sample can be obtained by removing a sample of cells from a subject, but can also be accomplished by using previously isolated cells (e.g. isolated by another person), or by performing the methods of the invention in vivo. Biological sample also refers to a sample of tissue or fluid isolated from an individual, including, but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, the biological samples can be prepared, for example biological samples may be fresh, fixed, frozen, or embedded in paraffin.

The term “tissue” is intended to include intact cells, blood, blood preparations such as plasma and serum, bones, joints, muscles, smooth muscles, and organs.

The term “treatment” refers to any treatment of a pathologic condition in a subject, particularly a human subject, and includes one or more of the following: (a) preventing a pathological condition from occurring in a subject which may have a predisposition to the condition but has not yet been diagnosed with the condition and, accordingly, the treatment constitutes prophylactic treatment for the disease or condition; (b) inhibiting the pathological condition, i.e. arresting its development, (c) relieving the pathological condition, i.e. causing a regression of the pathological condition; or (d) relieving the conditions mediated by the pathological condition.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—LINC00355 Binds to Menin to Regulate CDKN1B Expression and Promote Cell Proliferation

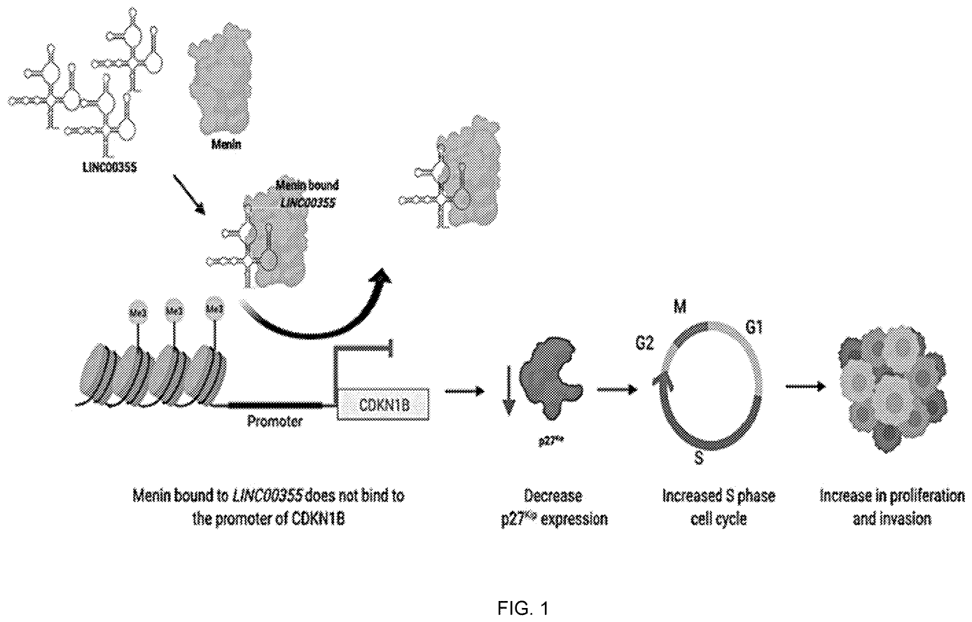

Previous research efforts identified 68 deregulated lncRNAs that were associated with late-stage relapse breast cancer as compared to primary breast cancer and early-stage relapse breast cancer (Eteleeb et al., NPJ Breast Cancer, 2022, 8, 49). The top up-regulated lncRNA LINC00355 (Fold change=7.21) was characterized (Eteleeb et al., NPJ Breast Cancer, 2022, 8, 49). LINC00355 is an annotated 1,878 nucleotide long lncRNA that was first identified in bladder cancer (Seitz et al., Scientific Reports, 2017, 7, 395) and more recently confirmed in other cancer types and diseases. LINC00355 promotes apoptosis and cell survival through its epigenetic regulation and post-transcriptional control (Alvarado-Hernandez et al., J Virol, 2022, 96, e0178221; Tan et al., Bioengineered, 2021, 12, 3410-3425; Luo et al., Journal of B.U.ON., 2021, 26, 1964-1969; Zhou et al., J Cancer, 2020, 11, 5641-5655; and Zhang et al., Antioxid Redox Signal, 2023, 39, 225-240). Genotype-tissue expression (GTEx) RNA sequencing shows that LINC00355 is weakly expressed in normal tissues and strongly expressed in whole blood, omentum, subcutaneous adipose, EBV-transformed lymphocytes, and testis. LncRNAs bind to proteins to regulate gene expression (Silva-Fisher et al., Nature Comm, 2020, 11, 2156; Wang et al., Molecular Cell, 2011, 43, 904-914; and Zhang et al., International Journal of Molecular Sciences, 2019, 20). Previous research efforts demonstrated that LINC00355 binds to the MENIN protein to regulate p27KIP1 expression by increasing histone H3 lysine 4 methylation (H3K4me3) on the CDKN1B promoter (Alrezk et al., Endocrine-related Cancer, 2017, 24, T195-T208; Pellegata et al., PNAS, 2006, 103, 15558-15563; Karnik et al., PNAS, 2005, 102, 14659-14664; Milne et al., PNAS, 2005, 102, 749-754; and Horiguchi et al., Clin Cancer Res, 2009, 15, 2620-2629) (FIG. 1). MENIN is encoded by the MEN1 gene that functions as an oncogene in a variety of cancer types including breast cancer (Dreijerink et al., Cell Reports, 2017, 18, 2359-2372 and Chandrasekharappa et al., Science, 1997, 276, 404-407). It is widely expressed in many normal tissues, including the pituitary, pancreas, and adrenal glands, and has a role in maintaining cellular homeostasis. MENIN and mixed lineage leukemia (MLL) have been found to co-regulate cell signaling by playing a role in regulating cyclin-dependent kinases p18 and p27KIP1 (Milne et al., PNAS, 2005, 102, 749-754 and Matkar et al., Trends Biochem Sci, 2013, 38, 394-402). This work provided evidence that LINC00355 regulated cellular proliferation by binding to the MENIN protein.

### Example 2—Linc00355 is Expressed in Long-Term Estrogen Deprived Cell Lines and Patient-Derived Xenograft Tumors

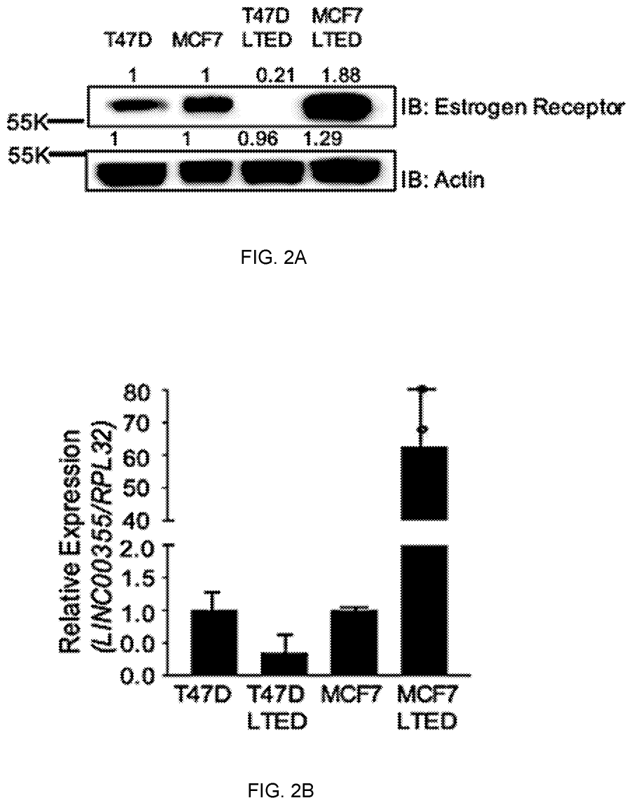

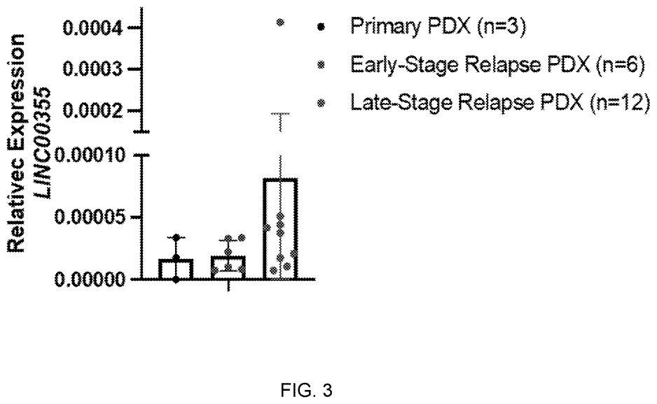

To understand how the LINC00355-MENIN interaction promotes cellular proliferation, two technologies were leveraged: long-term estrogen-deprived (LTED) cell lines (Eteleeb et al., NPJ Breast Cancer, 2022, 8, 49 and Li et al., Cell Reports, 2013, 4, 1116-1130), which have been widely used to study cellular adaptation to estrogen-deprived environments, and Washington University in St. Louis (WHIM) patient-derived xenograft (PDX) tumor lines, which retain genomic characteristics closely resembling their original human tumors (Ma et al., J Clin Invest, 2012, 122, 1541-1552 and McDonald et al., PNAS, 1989, 86, 3924-3927). Because MENIN has functions as a transcriptional regulator of Estrogen Receptor alpha (ERα+) (Dreijerink et al., Cell Reports, 2017, 18, 2359-2372 and Imachi et al., Breast Cancer Res Treat, 2010, 122, 395-407) and previous work implicates that LINC00355 has an association with ERα+ signaling (Eteleeb et al., NPJ Breast Cancer, 2022, 8, 49), LINC00355 expression in the LTED model system, which have been estrogen-deprived for more than three years was first assessed (Li et al., Cell Reports, 2013, 4, 1116-1130). The LTED model is present with either no ESR1 locus amplification as seen in the T47D LTED cell line (Fold=0.21) or amplification leading to increased ERα protein expression as seen in the MCF7 LTED cell line (Fold=1.88) as compared to their respective wild-type 51,55,56 (FIG. 2A). LINC00355 expression levels were 65-fold lower in T47D LTED cells than in wild-type T47D cells and 60-fold higher in MCF7 LTED cell than in wild-type MCF7 cells (FIG. 2B). Next, LINC00355 levels in ERα+ primary breast PDX tumor lines, early-stage relapse breast PDX tumor lines, and late-stage relapse PDX tumor lines were measured (FIG. 3). LINC00355 was found to be more highly expressed in late-stage relapse PDX lines as compared primary breast PDX lines (Fold=8) or early-stage relapse breast PDX lines (Fold=14, FIG. 3). This data mimics what was found in patient samples (Eteleeb et al., NPJ Breast Cancer, 2022, 8, 49). The variations in LINC00355 expression across LTED cells and PDX tumor models provides a unique opportunity to investigate the mechanisms driving these differences. This comparative analysis can identify cell line or tumor-specific regulatory networks that are targeted by LINC00355.

### Example 3—Identification of LINC00355 Binding Site on Menin Protein

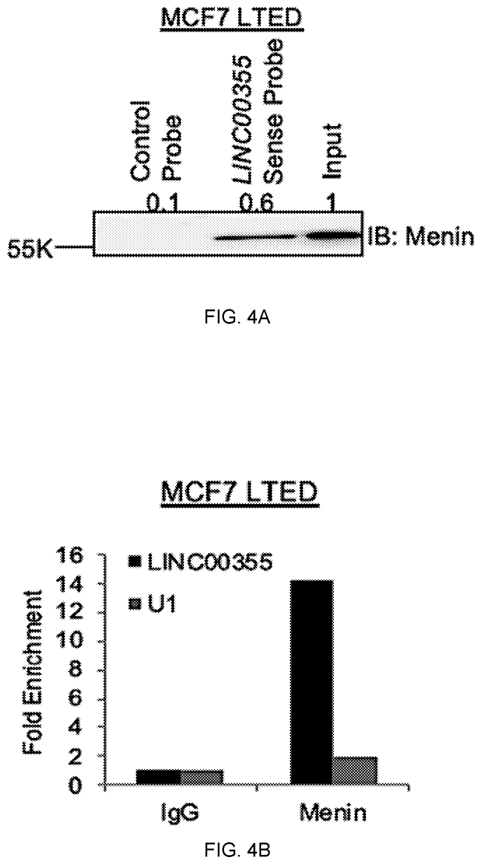

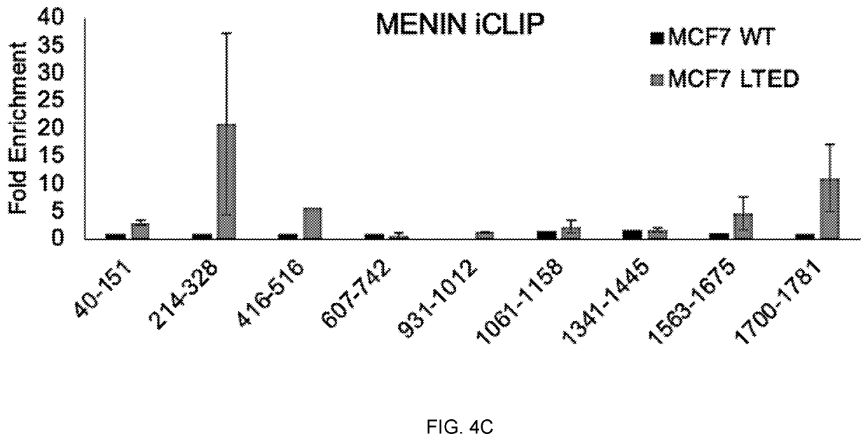

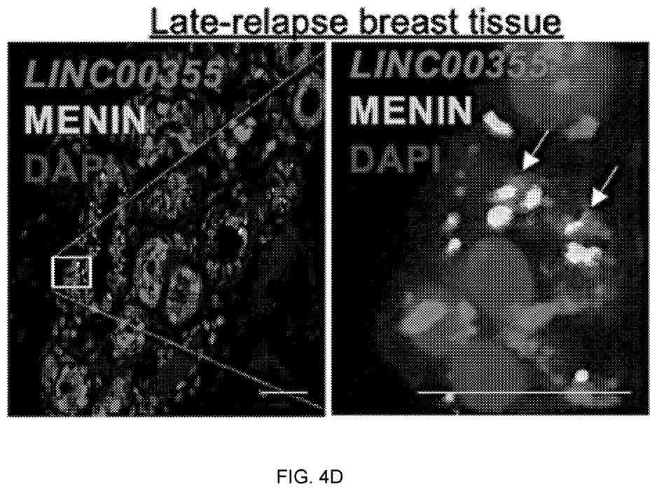

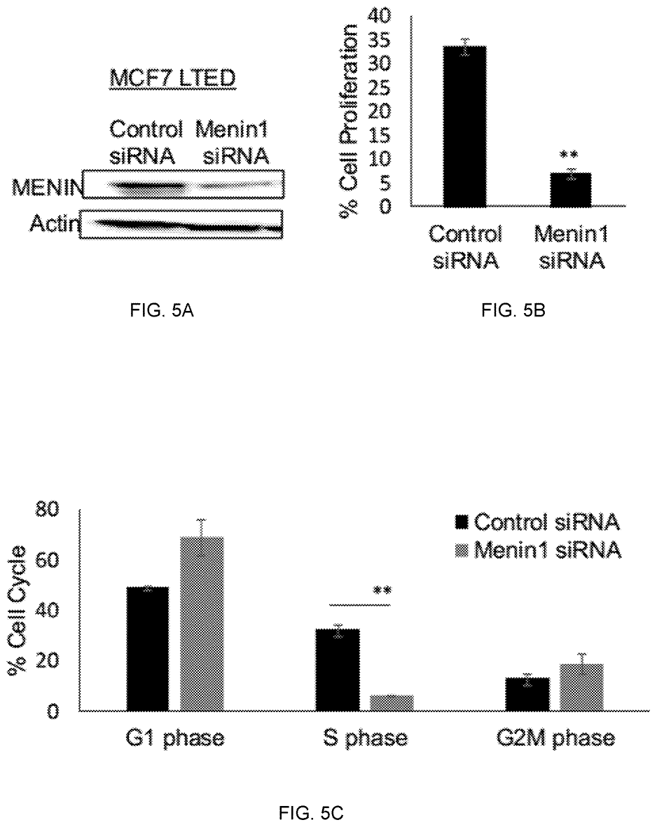

Previous research efforts provided evidence that LINC00355 bound to MENIN using RNA pulldown assays and RNA immunoprecipitation coupled with quantitative reverse transcription PCR (FIGS. 4A and 4B, and (Eteleeb et al., NPJ Breast Cancer, 2022, 8, 49)). Regions of LINC00355 that directly bind to MENIN were identified by performing individual-nucleotide resolution cross-linking immunoprecipitation (iCLIP) analysis, which uses UV cross-linking with immunoprecipitation to precisely map RNA-binding protein binding sites, coupled with RT-qPCR. In MCF7 LTED cells, RT-qPCR tiling primers spanning LINC00355 directly bind to MENIN at the 214-328 nucleotide region (Fold=20) and 1700-1781 nucleotide region (Fold=10), with less binding in wild-type MCF7 cells (FIG. 4C). Next, multiplex RNA-fluorescence in situ hybridization (RNA-FISH) was conducted with probes specific to LINC00355 in combination with immunohistochemistry with Menin antibodies to show co-detection of LINC00355 and MENIN in a late-relapse breast tumor (FIG. 4D). Results further show that reduced MENIN expression levels also decrease cellular proliferation and S-phase of the cell cycle (FIGS. 5A-5C).

### Example 4—Treating Cells with LINC00355 Locked Nucleic Acid Antisense Oligo Reduces Cellular Proliferation

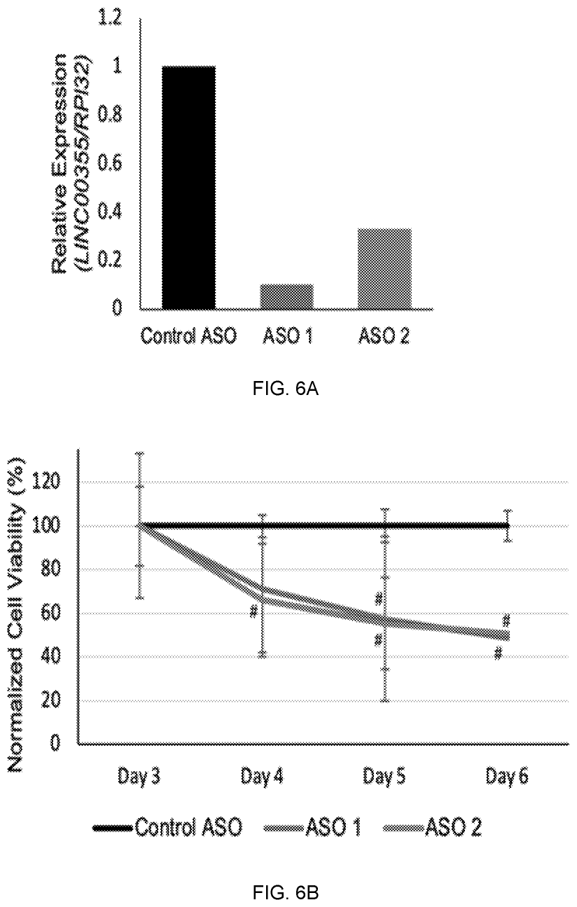

The role of LINC00355 in regulating cellular proliferation was investigated using two custom-designed in vitro-locked nucleic acid GapmeR antisense oligonucleotides (ASOs) that were specifically designed to silence LINC00355 expression in MCF7 LTED cell lines (FIG. 6A). A significant reduction in cell viability (p<0.0005) as measured by the CellTiter-Glo assay (Promega) was observed (FIG. 6B). Both ASOs resulted in more than 79% decrease in cell invasion compared to control ASOs (p<0.0005), indicating the functional importance of LINC00355 in cellular processes (Eteleeb et al., NPJ Breast Cancer, 2022, 8, 49). These findings demonstrate the potential of synthetic oligonucleotides to modulate LINC00355 expression and thereby reveal its associated effects on cell proliferation and invasion.

### Example 5—LINC00355-Menin Regulates CDK4/6 Activity

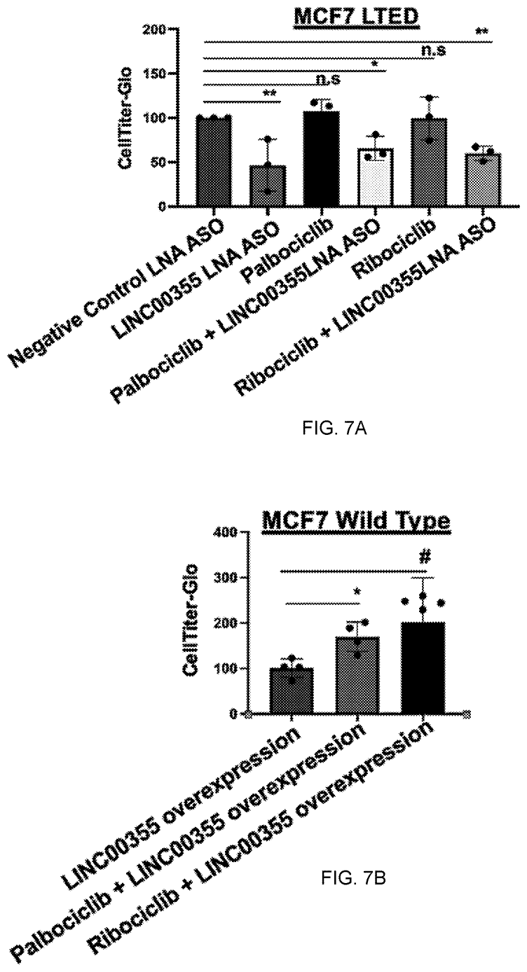

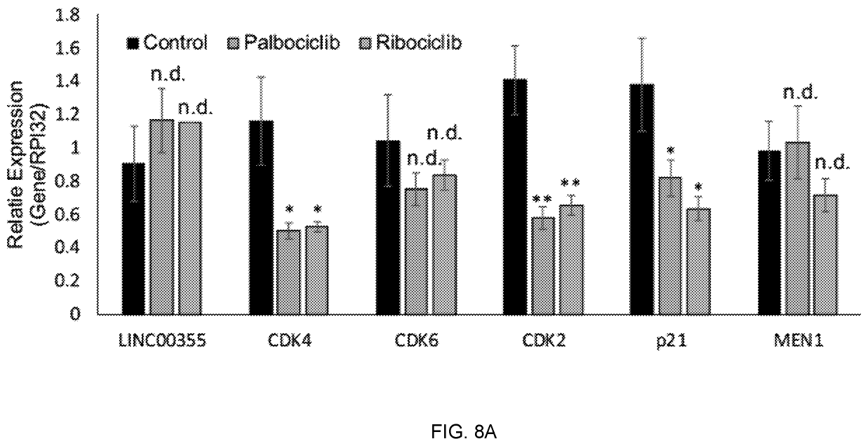

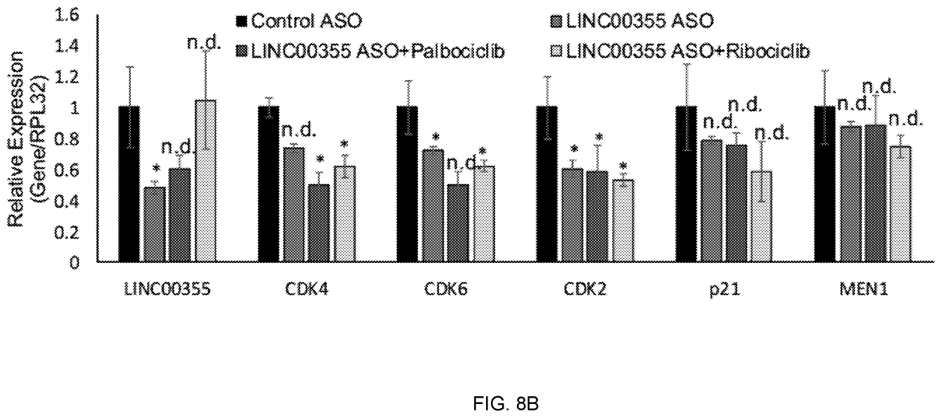

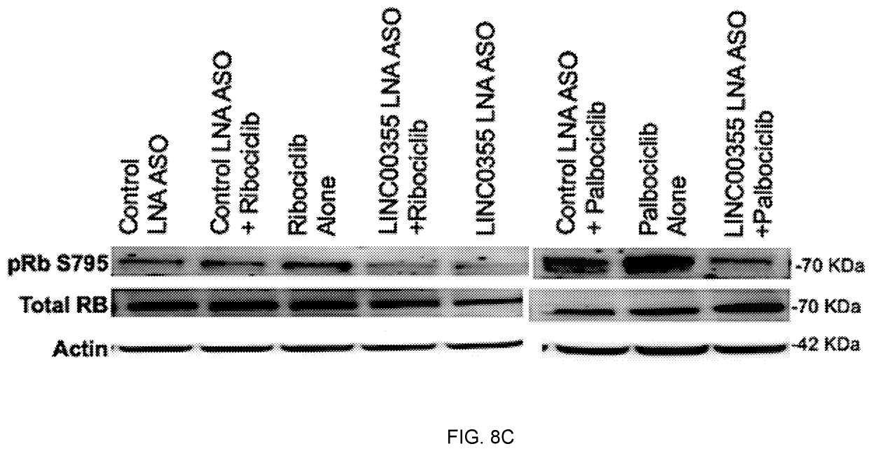

MENIN is a known modulator of the CDK4/6 pathway that has a role in balancing cell proliferation and growth arrest (Petrossian et al., Oncotarget, 2018, 9, 27736-27751 and Soto-Feliciano et al., Cancer Discov, 2023, 13, 146-169). The current disclosure provides evidence that LINC00355 regulates cell proliferation by binding to MENIN. A recent study reported that CDK4/6 small molecule inhibitors (CDK4/6i) regulate CDK4/6 activity in MCF7 wild-type cell line models (lida et al., Oncotarget, 2019, 10, 4907-4918 and Ji et al., Cell Death & Disease, 2020, 11, 760) although their effects in LTED cell models have not yet been assessed. Functionally, knockdown of LINC00355 using locked nucleic acid antisense oligonucleotides (LNA ASOs) significantly reduced tumor cell proliferation (FIG. 7A). Importantly, co-treatment with LINC00355-targeting LNA ASOs and the CDK4/6i, palbociclib or ribociclib, further suppressed proliferation, whereas LINC00355 overexpression increased proliferation and attenuated the effects of CDK4/6 inhibition (FIGS. 7A and 7B). This interaction was further investigated by performing RT-qPCR to analyze the expression of cell cycle-related genes, and observed that the expression levels of CDK4, p21, and the S-phase gene CDK2 (phosphorylates Rb59) were significantly lower when treated with CDK4/6i compared to controls (FIGS. 8A and 8B). Significant changes in the expression levels of CDK6 or MEN1 were not observed. Treating MCF7 LTED cells with LINC00355-targeted ASOs alone confirmed that the expression levels of LINC00355, CDK6, and CDK2 were reduced compared to controls. Further, treating MCF7 LTED cells with LINC00355-targeted ASOs combined with CDK4/6i reduced the expression levels of CDK4, CDK6, and CDK2. Consistently, combined LINC00355 knockdown and CDK4/6 inhibition reduced RB phosphorylation at serine 795, a canonical CDK4/6-dependent site (FIG. 8C).

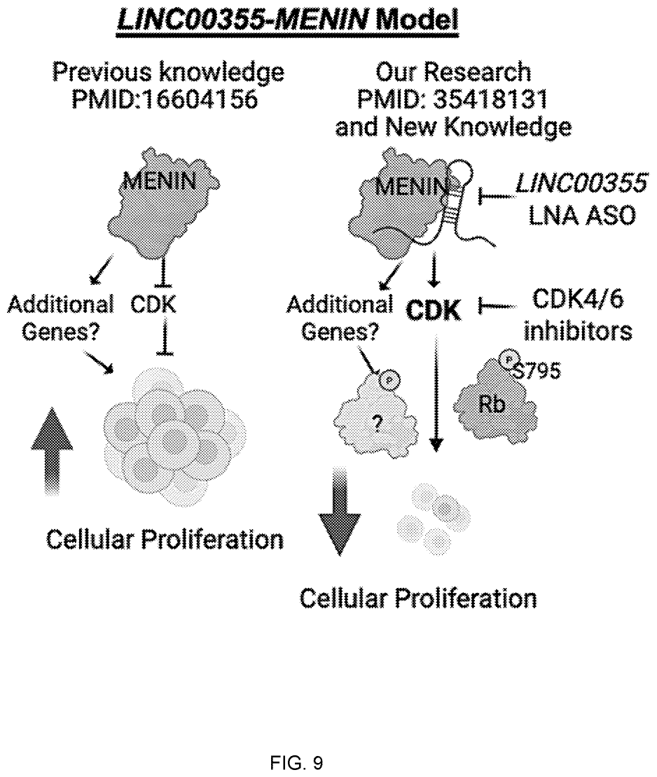

These data support that LINC00355 is highly expressed and specific to late-relapse breast cancer. In addition, LINC00355-MENIN interactions drive CDK4/6-dependent proliferation and LINC00355 also regulates MENIN-independent transcriptional programs (FIG. 9).

What is claimed is:

1. A composition comprising a locked nucleic acid-based antisense oligonucleotide (LNA ASO), wherein the locked nucleic acid-based antisense oligonucleotide targets LINC00355.

2. The composition of claim 1, wherein the locked nucleic acid-based antisense oligonucleotide comprises SEQ ID NO:3 or SEQ ID NO:4.

3. The composition of claim 1, further comprising a CDK4/6 inhibitor.

4. The composition of claim 3, wherein the CDK4/6 inhibitor is selected from the group consisting of Abemaciclib, Palbociclib, Ribociclib, or combinations thereof.

5. A pharmaceutical composition comprising the composition of claim 1, or a pharmaceutically acceptable salt thereof, and a pharmaceutically acceptable carrier.

6. The pharmaceutical composition of claim 5, wherein the pharmaceutically acceptable carrier is selected from the group consisting of a phosphate buffered saline solution, water, and a water/oil emulsion.

7. A method of preventing or treating cancer comprising administering a therapeutically effective amount of a composition comprising a locked nucleic acid-based antisense oligonucleotide (LNA ASO), wherein the locked nucleic acid-based antisense oligonucleotide targets LINC00355.

8. The method of claim 7, wherein the cancer is late relapse breast cancer.

9. The method of claim 7, wherein the locked nucleic acid-based antisense oligonucleotide comprises SEQ ID NO:3 or SEQ ID NO:4.

10. The method of claim 7, further comprising administering a CDK4/6 inhibitor.

11. The method of claim 10, wherein the CDK4/6 inhibitor is selected from the group consisting of Abemaciclib, Palbociclib, Ribociclib, or combinations thereof.

12. The method of claim 10, wherein administering the CDK4/6 inhibitor is prior to the administering of the therapeutically effective amount of the composition comprising the locked nucleic acid-based antisense oligonucleotide (LNA ASO) or after the administering of the therapeutically effective amount of the composition comprising the locked nucleic acid-based antisense oligonucleotide (LNA ASO).

13. The method of claim 10, wherein the CDK4/6 inhibitor and the therapeutically effective amount of the composition comprising the locked nucleic acid-based antisense oligonucleotide (LNA ASO) are administered simultaneously.

14. A method for decreasing LINC00355 lncRNA level in a cancer cell comprising administering a therapeutically effective amount of a composition comprising a locked nucleic acid-based antisense oligonucleotide (LNA ASO), wherein the locked nucleic acid-based antisense oligonucleotide targets LINC00355.

15. The method of claim 14, wherein the cancer is late relapse breast cancer.

16. The method of claim 14, wherein the locked nucleic acid-based antisense oligonucleotide comprises SEQ ID NO:3 or SEQ ID NO:4.

17. The method of claim 14, further comprising administering a CDK4/6 inhibitor.

18. The method of claim 17, wherein the CDK4/6 inhibitor is selected from the group consisting of Abemaciclib, Palbociclib, Ribociclib, or combinations thereof.

19. The method of claim 17, wherein administering the CDK4/6 inhibitor is prior to the administering of the therapeutically effective amount of the composition comprising the locked nucleic acid-based antisense oligonucleotide (LNA ASO) or after the administering of the therapeutically effective amount of the composition comprising the locked nucleic acid-based antisense oligonucleotide (LNA ASO).

20. The method of claim 17, wherein the CDK4/6 inhibitor and the therapeutically effective amount of the composition comprising the locked nucleic acid-based antisense oligonucleotide (LNA ASO) are administered simultaneously.
